(19) United States
(12) Patent Application Publication
(10) Pub. No.: US 2007/0043342 A1
(43) Pub. Date: Feb. 22, 2007

(54) Cryoprobe with reduced adhesion to frozen tissue, and cryosurgical methods utilizing same

(75) Inventor: Paul Kleinberger, Jerusalem (IL)

Correspondence Address: Martin Moynihan;PRTSI, Inc., P.O. Box 16446, Arlington, VA, 22215, US

(73) Assignee: Galil Medical Ltd., Yokneam (IL)

(21) Appl. No.: 11/204,175

(22) Filed: Aug. 16, 2005

(51) Int. Cl. A61B 18/02

(52) U.S. Cl. 606/21; 606/23

(57) ABSTRACT

The present invention relates to devices and methods for cryosurgery. More particularly, the present invention relates to a cryoprobe which does not form strong adhesive or mechanical bonds with body tissues when such tissues are frozen by cooling action of the probe. Embodiments of the present invention include a cryoprobe having a distal cooling module with an outer surface layer of non-polar molecules, a probe having a distal cooling module with a microscopically smooth outer surface, and a cryoprobe comprising a mechanism for coating a distal cooling module thereof with non-polar lubricant during movement of the cryoprobe within body tissues of a patient. Also presented are methods utilizing disclosed cryoprobes to facilitate cryosurgery and to enhance accuracy of cryoablation of user-selected cryoablation targets.

### FIELD AND BACKGROUND OF THE INVENTION

The present invention relates to devices and methods for cryosurgery. More particularly, the present invention relates to a cryoprobe to which body tissues do not tend to adhere strongly when frozen by cooling action of the probe. Embodiments of the present invention include a cryoprobe having a cooling module with an outer surface layer of non-polar molecules, a cryoprobe having a cooling module with a microscopically smooth outer surface, and a cryoprobe comprising a mechanism for coating a cooling module thereof with non-polar lubricant during movement of the cryoprobe within body tissues of a patient. Also presented are methods utilizing such probes to facilitate cryosurgery and to enhance accuracy of cryoablation of a user-selected cryoablation target.

In an increasingly popular therapeutic technique, cryoprobes are used to ablate pathological tissues by cooling those tissues to cryoablation temperatures. One issue complicating use of cryoprobes is a tendency of body tissues to adhere to cryoprobes when those tissues freeze during cryosurgery. In current practice according to methods of prior art, once an inserted cryoprobe is used to cool tissue and that cooled tissue freezes, the inserted cryoprobe cannot be displaced within the patient's body nor removed from that body, because the probe, firmly adhering to the body tissues, cannot be moved without tearing those tissues, if it can be moved at all. Generally speaking, tissues adjacent to a cryoprobe must be thawed before an inserted and cooled cryoprobe can be moved to a new location within a patient's body. Similarly, tissues adjacent to a cryoprobe must be thawed before a cooling cryoprobe can be removed from a patient's body on completion of a surgical intervention.

Adherence of tissues to cooling cryoprobes is an expected integral part of cryosurgery, and constitutes a problem to be overcome during removal and/or repositioning of probes. Many contemporary surgical procedures require movement of cryoprobes following freezing. Preferred treatment protocols often call for placement of a probe at a first site, cooling of tissues at that first site, then displacement of the probe to a second site, as cryosurgeons seek ever better ways to accurately tailor the three-dimensional shape of cryoablation volumes created by their probes to the three-dimensional shape of their intended organic cryoablation targets. Thus, rapid displacement of cryoprobes during cryosurgery is a requirement of some treatment protocols.

Rapid removal of cryoprobes from a body following cryosurgery is a practical requirement of most cryosurgical interventions, since waiting around for tissues to naturally thaw at the end of an intervention is not an efficient use of time for a busy surgeon. Clearly, improved devices and methods which speed up surgical procedures without deleterious side-effects would be highly desirable. Cryoprobes which could be removed easily at termination of surgical procedures would contribute to the practical efficiency of those procedures.

Breast surgery is an example of a type of surgery in which rapid removal of a cryosurgery needle following cryosurgery is advantageous to the patient as well as to the surgeon. Since in breast cryosurgery a patient typically undergoes local and not general anesthetic, devices and methods enabling to shorten the time during which cryoneedles are inserted in the breast would be welcomed by patients as well as by surgical practitioners.

Thus, there is a widely recognized need for, and it would be highly advantageous to have, a cryoprobe which can rapidly be displaced within body tissues after being used to freeze tissues. There is further a widely recognized need for, and it would be highly advantageous to have, a cryoprobe which can rapidly be removed from a body after being used to freeze tissues.

In some surgical contexts adherence of tissues to cryoprobes can be dangerous as well as merely inconvenient. Adherence of delicate and vulnerable tissues to a cryoprobe held in the hands of a surgeon can constitute a significant danger in certain surgical interventions, because delicate adhering tissues can inadvertently be torn or otherwise mechanically damaged. Adherence of moving tissues (e.g. heart muscle) to a hand-held or mechanically immobilized cryoprobe can cause mechanical damage to tissues as well.

Cryosurgical therapy for cardiac arrhythmia is an example of a surgical context in which a cryoprobe having a reduced tendency to adhere to freezing tissues would be particularly advantageous. U.S. patent application Ser. No. 10/311,315 by Zvuloni describes a treatment protocol wherein adhesion of a probe to tissue of a beating heart is desired at a certain phase of the procedure, to immobilize the cryoprobe/tissue interface while testing whether treatment at a given position will cure arrhythmia. However, as thawing begins and portions of frozen heart muscle in proximity to an adhering treating cryprobe begin to thaw and to beat, residual adhesion of cryoprobe to tissue at that time can cause tearing of delicate heart tissue or delicate blood vessel tissue of the pulmonary vein ostium.

Thus, there is a widely recognized need for, and it would be highly advantageous to have, a cryoprobe which adheres relatively weakly to frozen tissue, and is easily and rapidly freed during thawing. Such a probe can protect delicate tissues otherwise endangered by cryoablation practiced according to methods of prior art.

In a variety of applications, it would be advantageous to have a cryoprobe operable to freeze tissues while moving. U.S. Pat. No. 6,875,209 to Zvuloni et al. describes treatment of arterial plaque using a cooling expandable balloon catheter. U.S. Pat. No. 6,875,209 to Zvuloni et al. is incorporated herein by reference. The cryoplasty treatment described therein requires minimal cooling, and is preferably executed rapidly, to avoid prolonged strangling of blood supply within a treated blood vessel. For this and similar treatment applications, there is a widely felt need for, and it would be highly advantageous to have, an expandable balloon catheter operable to move within a blood vessel or other body conduit while performing a cooling function, even when that cooling function causes cooling of tissues is to below-freezing temperatures.

In a related area, U.S. patent application Ser. No. 11/066,294 by Zvuloni et al. teaches a cryosurgery method in which a cryoablation volume created by cooling a plurality of cryoprobes is exactly tailored to a three-dimensional cryoablation target by differential cooling, wherein central portions of a cryoablation target are strongly cooled by a first set of cryoprobes, and peripheral portions of that target are weakly cooled by a second set of cryoprobes, thereby controlling and limiting damage to tissues adjacent to, but not within, a user-defined three-dimensional cryoablation target. More convenient and more efficient methods for accomplishing strong cooling of central portions of a cryoablation target and weak cooling of peripheral portions of a cryoablation target would be possible utilizing cryoprobes operable to move within tissue during, or immediately following, cooling that tissue to below-freezing temperatures.

Thus there is a widely recognized need for, and it would be highly advantageous to have, a cryosurgery method utilizing moving cryoprobes to facilitate exact tailoring of a cryoablation volume to a user-selected cryoablation target, by controlled differential cooling of selected internal portions of a cryoablation target using a cryoprobe operable to cool while moving through body tissues. To utilize those methods, and for a variety of similar cryosurgical treatments, there is a widely felt need for, and it would be highly advantageous to have, a cryoprobe operable to move within body tissues while performing a cooling function, even when that cooling function causes cooling of tissues is to below-freezing temperatures.

The problem of adherence of cryoprobes to tissues has generally been solved by supplying cryoprobes with heating mechanisms along with their cooling mechanisms, enabling the probes to thaw tissues as well as freeze them. Cryoprobes that cool using Joule-Thomson cooling (i.e., cooling by expansion of a high-pressure cooling gas such as argon) are often also operable to heat by Joule-Thomson heating (i.e., by expansion of a high-pressure heating gas such as helium), and cryosurgery systems are equipped to selectively supply both heating gas and cooling gas. Various other methods for heating cryoprobes are also known in the art.

The role of heating as a component of cryoablation therapies is currently under discussion among cryosurgical practitioners. Heated thawing is often considered a necessary part of the cryoablation process, yet in discussions comparing the therapeutic advantages and disadvantages of natural thawing as opposed to heating thawing, the disadvantage of natural thawing most often cited is the long delays required before inserted cryoprobes can be moved or removed, when natural thawing is used. Thus, cryoprobes operable to be removed following cryoablation cooling without prior thawing of tissues might provide a therapeutic as well as a practical advantage. Thus, there is a widely recognized need for, and it would be highly desirable to have, a cryoprobe which makes it practical for a surgeon to use natural thawing in cryoablation procedures.

Equipping cryoprobes with heating mechanisms adds to the complexity and cost of cryosurgical systems, and adds also to the complexity of operating procedures using those probes. Heating probes prior to moving them can also be somewhat time-consuming. Thus, there is a widely recognized need for, and it would be highly advantageous to have, a cryoprobe which does not require heating, or which requires only minimal heating, before being moved after being used to freeze tissue.

With reference to certain techniques and technologies used within embodiments presented hereinbelow, note is taken that use has been made in various household appliances of microscopically smooth surfaces designed to minimize adhesion of foreign matter to those surfaces. For example, Toto Ltd. of Japan has produced a toilet bowl having an interior surface smoothed “on a nanometer scale”, which surface, by virtue of its nearly perfect smoothness at a microscopic level, is resistant to adhesion by dirt and bacteria. Cryoprobes known to prior art have external surfaces which, though they appear smooth to the naked eye, are not in fact smooth on a microscopic (e.g. nanometer) scale, and which therefore present geometrically complex surfaces such as concavities (at the microscopic level) within which ice crystals may form and from which those ice crystals cannot easily be dislodged. Therefore there is a widely felt need for, and it would be highly advantageous to have, a cryoprobe presenting a distal cooling surface having a geometrically simple and microscopically smooth surface, where that surface comes in contact with freezing tissues.

### SUMMARY OF THE INVENTION

According to one aspect of the present invention there is provided a cryoprobe with reduced tendency to adhere to frozen tissues, comprising one of a group consisting of:

(a) a cooling module having a microscopically smooth external surface;

(b) a cooling module having an external surface comprising non-polar material;

(c) a cooling module coated with a substantially non-polar substance having lubricating qualities at room temperature and when cooled to below-freezing temperatures;

(d) an external orifice through which a biocompatible non-polar substance, delivered to the orifice through communicating with the orifice, may be extruded during movement of the cryoprobe within a body of a patient; and

(e) a mechanical attachment operable to impart small repetitive motions to an inserted cryoprobe while the inserted cryoprobe is cooled to below-freezing temperatures.

In a preferred embodiment the cryoprobe has a microscopically smooth external surface comprising non-polar material and an external orifice communicating with an internal lumen through which a non-polar substance may be extruded during movement of the cryoprobe within a body of a patient. Preferably the cryoprobe further comprises a mechanical attachment operable to impart small repetitive motions to an inserted cryoprobe while the inserted cryoprobe is cooled to below-freezing temperatures. These motions may include longitudinal movements, rotational movements, and vibratory movements.

According to further features in preferred embodiments of the invention described below, the cryoprobe comprises a cooling module having an external surface which comprises non-polar material, wherein exposed portions of surface molecules of the external surface are predominantly non-polar. The surface layer may comprise Teflon®.

According to further features in preferred embodiments of the invention described below the cryoprobe comprises an orifice so designed and constructed that the biocompatible non-polar substance extruded through the orifice while the cryoprobe is inserted into a body of a patient at least partially coats an external surface of a cooling module of the cryoprobe. Optionally, the cryoprobe also comprises and orifice so designed and constructed that the biocompatible non-polar substance extruded through the orifice while the cryoprobe is withdrawn from a body of a patient at least partially coats an external surface of a cooling module of the cryoprobe. The orifices may be positioned distally with respect to at least a portion of a cooling module of the cryoprobe and/or or proximally with respect to at least a portion of a cooling module of the cryoprobe.

According to another aspect of the present invention there is provided a cryoprobe with reduced tendency to adhere to frozen tissues, comprising an external surface designed and constructed to form at most weak chemical and mechanical bonding with ice crystals that form in proximity to the cryoprobe when a cooling module of the cryoprobe is cooled to below-freezing temperatures.

According to yet another aspect of the present invention there is provided a system for cryoablation of a user-selected cryoablation target, comprising:

(a) a cryoprobe navigable within tissues of a body while being cooled to below-freezing temperatures;

(b) a first data source providing real-time data relating to positioning of the cryoprobe with respect to the user-selected cryoablation target;

(c) a second data source providing real-time data relating to temperature of the cryoprobe; and

(d) a controller operable to calculate preferred operating parameters for the cryoprobe as a function of data from the first and second data sources.

Preferably, the system further comprises a servomechanism operable to displace the cryoprobe within a body of a patient according to commands issued by the controller, and a cryogen supply operable to supply a controlled amount of cryogen to the cryoprobe according to commands issued by the controller. The cryogen is preferably a compressed gas and the cryogen supply is operable to control flow of gas supplied by the gas supply to the cryoprobe.

Preferably, the system further comprises:

a) a servomechanism operable to displace the cryoprobe within a body of a patient according to commands issued by the controller; and

(b) a cooling gas supply operable to supply cooling gas to the cryoprobe, comprising a source of high-pressure cooling gas and gas-supply controller operable to control flow of gas from the gas supply to the cryoprobe according to commands issued by the controller.

According to further features in preferred embodiments of the invention described below the controller is operable to calculate and command speed of movement of the cryoprobe within a body of a patient as a function of temperature of the cryoprobe and further as a function of position of the cryoprobe in relation to the user-selected cryoablation target. The controller is also operable to calculate and command a rate of cooling of the cryoprobe as a function of position of the cryoprobe with respect to a user-selected cryoablation target, speed of movement of the cryoprobe, and detected temperature of the cryoprobe.

According to still another aspect of the present invention there is provided a method for cryotreatment of a patient, comprising:

(a) inserting into tissues of a body of the patient a cryoprobe which comprises an external surface designed and constructed to form at most weak bonds with ice crystals that form in proximity to the cryoprobe when a cooling module of the cryoprobe is cooled to below-freezing temperatures; and

(b) displacing the cryoprobe within the tissues while cooling the cryoprobe to below freezing temperatures.

According to further features in preferred embodiments of the invention described below the method further comprises using a control module to calculate cooling parameters and movement parameters for the cryoprobe based on a plurality of data streams, which data streams may include data relating to temperature of the cryoprobe and data relating to position of the cryoprobe with respect to a user-selected cryoablation target.

According to still another aspect of the present invention there is provided a balloon catheter sized for insertion into a body conduit and operable to cool a wall of said body conduit, comprising one of a group consisting of:

(a) a cooling module having a microscopically smooth external surface;

(b) a cooling module having an external surface comprising non-polar material;

(c) a cooling module coated with a substantially non-polar substance having lubricating qualities at room temperature and when cooled to below-freezing temperatures;

(d) an external orifice through which a biocompatible non-polar substance, delivered to said orifice through communicating with said orifice, may be extruded during movement of said cryoprobe within a body of a patient; and

(e) a mechanical attachment operable to impart small repetitive motions to an inserted cryoprobe while said inserted cryoprobe is cooled to below-freezing temperatures.

The present invention successfully addresses the shortcomings of the presently known configurations by providing a cryoprobe which can rapidly be removed from a body, or can rapidly be displaced within a body, after being used to freeze body tissues.

The present invention further successfully addresses the shortcomings of the presently known configurations by providing a cryoprobe which adheres relatively weakly to frozen tissue, and is easily and rapidly freed during thawing.

The present invention further successfully addresses the shortcomings of the presently known configurations by providing an expandable balloon catheter operable to move within a blood vessel or other body conduit while performing its cooling function, even when that cooling function causes cooling of tissues to below-freezing temperatures.

The present invention further successfully addresses the shortcomings of the presently known configurations by providing a cryoprobe operable to move within body tissues while performing a cooling function, even when that cooling function causes cooling of tissues is to below-freezing temperatures.

The present invention further successfully addresses the shortcomings of the presently known configurations by providing a cryosurgery method utilizing moving cryoprobes to facilitate exact tailoring of a cryoablation volume to a user-selected cryoablation target.

The present invention further successfully addresses the shortcomings of the presently known configurations by providing a cryoprobe which makes it practical for a surgeon to use natural thawing in cryoablation procedures.

The present invention successfully addresses the shortcomings of the presently known configurations by providing a cryoprobe which requires no heating system or a less powerful heating system than those necessitated in cryoprobes currently known.

The present invention further successfully addresses the shortcomings of the presently known configurations by providing a cryoprobe presenting a distal cooling surface having a geometrically simple and microscopically smooth surface at the contact interface between that surface and freezing body tissues.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified schematic of a cryoprobe according to methods of prior art;

FIG. 2 is a simplified schematic of a portion of a cryoprobe having a non-polar exterior surface, according to an embodiment of the present invention;

FIG. 3 is a simplified schematic of a cryoprobe having a microscopically smooth exterior surface, according to an embodiment of the present invention;

FIG. 4 is a simplified schematic of a cryoprobe having a lumen for transporting a non-polar lubricating substance to an external orifice of the probe, according to an embodiment of the present invention;

FIG. 5 is a simplified flow chart of a method of cryotherapy utilizing a non-polar lubricating substance on a cryoprobe, according to an embodiment of the present invention;

FIG. 6 is a flow chart of a procedure for accurately delimiting a cryoablation volume during cryoablation of a cryoablation target in a body of a patient, according to an embodiment of the present invention; and

FIG. 7 is a simplified schematic of a system for cryotherapy incorporating a servomotor and a non-polar lubricating substance source, according to an embodiment of the present invention.

### DESCRIPTION OF THE PREFERRED EMBODIMENTS

The present invention is of cryoprobes operable to cool body tissues to below-freezing temperatures without thereby creating strong bonding between frozen tissues and a cooling surface of the cryoprobe. Specifically, cryoprobes here disclosed enable to displace cryoprobes during cryosurgery without thawing of tissue or with only minimal thawing of tissue. Cryoprobes here disclosed also enable to freeze tissues using a moving cryoprobe, thereby enabling accurate tailoring of a cryoablation volume to a cryoablation target by use of moving cooling cryoprobes.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

To enhance clarity of the following descriptions, the following terms and phrases will first be defined:

The phrase “heat-exchanging configuration” is used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure.

The phrase “Joule-Thomson heat exchanger” as used herein refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice, referred to herein as a “Joule-Thomson orifice”, through which gas passes from the first, higher pressure, region of the device to the second, lower pressure, region of the device. A Joule-Thomson heat exchanger may further include a heat-exchanging configuration, for example a heat-exchanging configuration used to cool gasses within a first region of the device, prior to their expansion into a second region of the device.

The phrase “cooling gasses” is used herein to refer to gasses which have the property of becoming colder when passed through a Joule-Thomson heat exchanger. As is well known in the art, when gasses such as argon, nitrogen, air, krypton, CO₂, CF₄, and xenon, and various other gasses pass from a region of higher pressure to a region of lower pressure in a Joule-Thomson heat exchanger, these gasses cool and may to some extent liquefy, creating a cryogenic pool of liquefied gas. This process cools the Joule-Thomson heat exchanger itself, and also cools any thermally conductive materials in contact therewith. A gas having the property of becoming colder when passing through a Joule-Thomson heat exchanger is referred to as a “cooling gas” in the following.

The phrase “heating gasses” is used herein to refer to gasses which have the property of becoming hotter when passed through a Joule-Thomson heat exchanger. Helium is an example of a gas having this property. When helium passes from a region of higher pressure to a region of lower pressure, it is heated as a result. Thus, passing helium through a Joule-Thomson heat exchanger has the effect of causing the helium to heat, thereby heating the Joule-Thomson heat exchanger itself and also heating any thermally conductive materials in contact therewith. Helium and other gasses having this property are referred to as “heating gasses” in the following.

As used herein, a “Joule Thomson cooler” is a Joule Thomson heat exchanger used for cooling. As used herein, a “Joule Thomson heater” is a Joule Thomson heat exchanger used for heating.

The terms “ablation temperature” and “cryoablation temperature”, as used herein, relate to the temperature at which cell functionality and structure are destroyed by cooling. According to current practice temperatures below approximately −40° C. are generally considered to be ablation temperatures.

The terms “freezing temperature” and “freezing temperatures” refer to temperatures at which tissues of a body freeze. Freezing temperature is of course in the vicinity of 0° C., but will vary slightly depending on the exact composition of the tissues being frozen. Temperatures “below freezing temperatures” are of course temperatures below “freezing temperatures.”

The term “ablation volume”, as used herein, is the volume of tissue which has been cooled to ablation temperatures by one or more cryoprobes.

The terms “ablation target”, “cryoablation target”, and “three-dimensional cryoablation target” related to a user-defined volume which that user desires to cryoablate. An ablation target as defined by a surgeon will typically include a lesion which the surgeon wishes to destroy, and some margin of presumably healthy tissue also to be ablated as a precaution against any inaccuracies of diagnosis or procedure. For example, a surgeon might be expected to choose a broad margin of safety around a tumor known to be malignant, and a narrower margin of safety around a growth known to be benign. A lesion and a user-selected safety margin typically constitute a user-defined cryoablation target. It is to be noted that the term “user-defined cryoablation target” is to be understood as including cryoablation targets defined by an automated (algorithmic) process at the request of a human user.

As used herein, the term “high-pressure” as applied to a gas is used to refer to gas pressures appropriate for Joule-Thomson cooling of cryoprobes. In the case of argon gas, for example, “high-pressure” argon is typically between 3000 psi and 4500 psi, though somewhat higher and lower pressures may sometimes be used.

It is expected that during the life of this patent many relevant cryoprobes will be developed, and the scope of the term “cryoprobe” is intended to include all such new technologies a priori.

As used herein the term “about” refers to ±10%.

In discussion of the various figures described hereinbelow, like numbers refer to like parts.

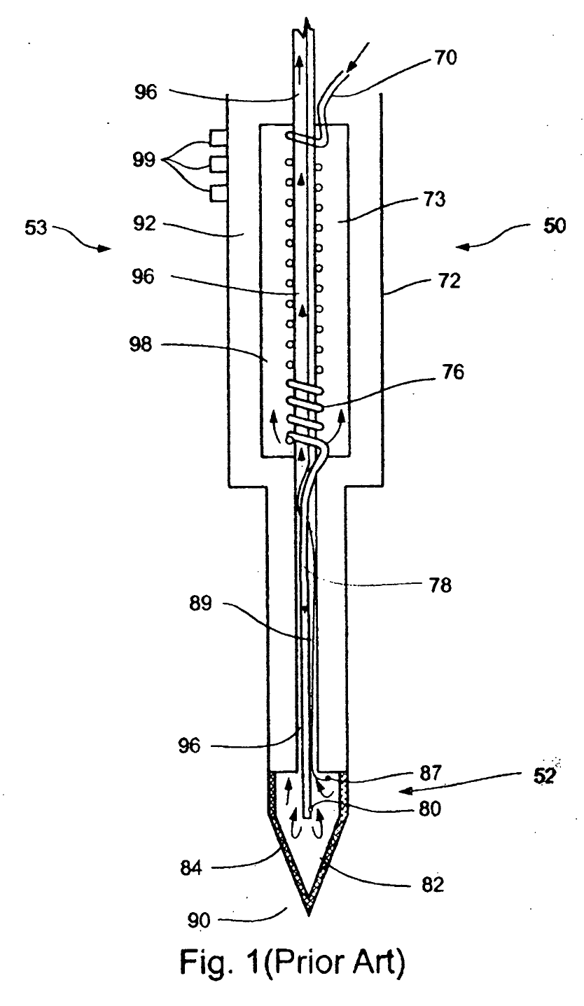

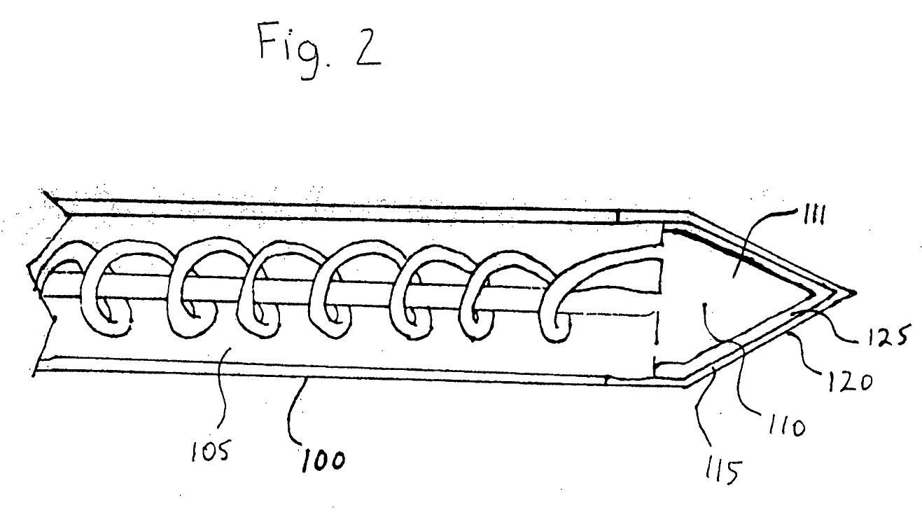

For purposes of better understanding the present invention, as illustrated in FIGS. 2-7 of the drawings, reference is first made to the construction and operation of a conventional (i.e., prior art) cryoprobe as illustrated in FIG. 1.

FIG. 1 presents a simplified schematic of a cryoprobe utilizable to affect cryoablation, according to a typical configuration of prior art.

As shown in FIG. 1, a cryoprobe 53 has an operating tip 52 including a Joule-Thomson cooler for freezing a patient's tissue, and a holding member 50 for holding by a surgeon. Operating tip 52 includes at least one passageway 78 extending therethrough for providing gas of high pressure to orifice 80 located at the end of operating tip 52, orifice 80 being for passage of high pressure gas therethrough, so as to cool operating tip 52 and produce an ice-ball at its end 90. Gases that may be used for cooling, referred to herein as “cooling gasses”, include, but are not limited to, argon, nitrogen, air, krypton, CO₂, CF₄, xenon, and N₂O.

When a high pressure cooling gas such as argon expands through orifice 80, it cools and may liquefy so as to form a cryogenic pool within chamber 82 of operating tip 52. The cooled gas and/or cryogenic pool effectively cools surface 84 of operating tip 52. Surface 84 of operating tip 52 is preferably made of a heat conducting material such as metal so as to enable the formation of an ice-ball at end 90 thereof. Deep cooling of tissues within an ice-ball effects cryoablation of those tissues.

Alternatively, a high-pressure gas such as helium may be used for heating operating tip 52 via a reverse Joule-Thomson process. Gasses, such as helium, having this property are referred to herein as “heating gasses.” When a high-pressure heating gas expands through orifice 80 it heats chamber 82, thereby heating surface 84 of operating tip 52. Heating of a cryoprobe is useful in that it enables treatment by cycles of cooling-heating, and is further useful during extraction of a cryoprobe from tissue which has been frozen, since melting the contact interface between a probe and frozen body tissues prevents sticking of the probe to the tissue when the probe is extracted from the patient's body. Heating of a cryoprobe also enables fast extraction, when desired.

Operating tip 52 includes at least one evacuating passageway 96 extending therethrough for evacuating gas from operating tip 52 to the atmosphere.

As shown FIG. 1, holding member 72 (or, alternatively, some other portion of probe 53) may include a preliminary heat-exchanging configuration 73 for pre-cooling gas flowing through passageway 78. Specifically, the upper portion of passageway 78 may be in the form of a spiral tube 76 wrapped around evacuating passageway 96, the spiral tube being accommodated within a chamber 98. Thus, expanded cooling gas evacuated through passageway 96 may pre-cool incoming cooling gas flowing through spiral tube 76. Similarly, expanded heating gas evacuated through passageway 96 may pre-heat incoming heating gas flowing through spiral tube 76.

As further shown in FIG. 1, holding member 72 may include an insulating body 92 for thermally insulating heat-exchanging configuration 73 from the external environment.

Furthermore, operating tip 52 may include at least one thermal sensor 87 for sensing the temperature within chamber 82, the wire 89 of which extending through evacuating passageway 96 or a dedicated passageway (not shown).

In addition, holding member 72 may include a plurality of switches 99 for manually controlling operation of probe 53 by a surgeon. Such switches may provide functions such as on/off, heating, cooling, and predetermined cycles of heating and cooling by selectively and controllably communicating incoming passageway 70 with an appropriate external gas container including a cooling or a heating gas.

Attention is now drawn to FIG. 2, which presents a simplified schematic of a portion of a cryoprobe having a non-polar external surface designed and constructed to minimize adhesion to frozen tissue, according to an embodiment of the present invention.

FIG. 2 presents a cryoprobe 100 which comprises a shaft 105 and a cooling module 110. With respect to its cooling features, cryoprobe 100 may constructed along the lines of cryoprobe 53 presented in FIG. 1, or may be constructed according to any other cryoprobe design enabling a cryoprobe to cool tissues to cryoablation temperatures. Thus, cooling module 110 may be constructed as described for operating tip 52 of cryoprobe 53, or may be constructed according to alternative methods of cryoprobe construction. In particular, cooling module 110 may be an evaporative cooling module 111, operative to cool cryoprobe 100 by evaporation of a liquefied gas such as liquid nitrogen, liquefied N₂O, liquefied CO₂, or a similar cryogenic fluid.

Cryoprobe 100 is designed and constructed to reduce bonding between external surfaces of cryoprobe 100 and ice formed in body tissues frozen by cooling action of cryoprobe 100. Cryoprobe 100 has an outer layer 115 which comprises a surface 120 of non-polar molecules at positions where cooling portions of cryoprobe 100 are in contact with body tissues when cryoprobe 100 is active in cooling. As discussed in the background section hereinabove, tissues frozen by cryoprobes constructed according to methods of prior art typically strongly adhere to cryoprobes which freeze them. Cryoprobe 100 is designed to reduce such adhesion.

Thus, layer 115 comprises a non-polar exterior surface 120. Layer 115 may be composed of non-polar molecules, or layer 115 may comprise a complex construction whose exposed surface face 120 is predominantly non-polar. Surface 120, being non-polar, will show a reduced tendency to form bonds with water molecules, and consequently will have a reduced tendency to form bonds with ice crystals and of frozen water and frozen tissue. Consequently body tissues will have a reduced tendency to adhere to cryoprobe 100 as those tissues are frozen by cooling action of probe 100.

Layer 115 covers at least a portion, and preferably all, of cooling module 110, and may additionally cover all or a portion of shaft 105. Thus, at surfaces wherein portions of probe 100 operable to cool probe 100 to cryoablation temperatures interface with (i.e. are contiguous to) moist body tissues, probe 100 presents a substantially non-polar interface, thus reducing tendencies of moist freezing tissue to bond to probe 100.

Layer 120 may be, for example, comprise Teflon® applied as a coating to cryoprobe 100 using methods well known in the art. Other non-polar or hydrophobic materials may be similarly used.

Many non-polar materials are poor conductors of heat, at least as compared to steel or other heat-conducting metals and similar materials of which cooling modules of cryoprobes are typically constructed. If, as may be the case, material comprising layer 115 is a poor conductor of heat, layer 115 will preferably be thin. Efficient transfer of heat between cooling module 110 and adjacent body tissues is generally a design goal for any cryoprobe, consequently coating a cryoprobe with an insulating layer might tend to be counter-productive. However, it may be noted that insulating properties of materials forming layer 115 depend on thickness of layer 115, whereas the non-polar character of surface 120 depends only on actual surface characteristics of exposed portions of surface molecules comprising surface 120. Consequently, constructing layer 115 as a thin layer, optionally only a few molecules thick, will enable surface 120 to present a non-polar exterior interface surface while yet transferring heat between tissues and probe with acceptable efficiency. Layer 115 may comprise the entire outside wall of cooling module 110, or may optionally be an external layer over an internal wall layer 125 comprised of a strong material with high heat transference, such as a metal.

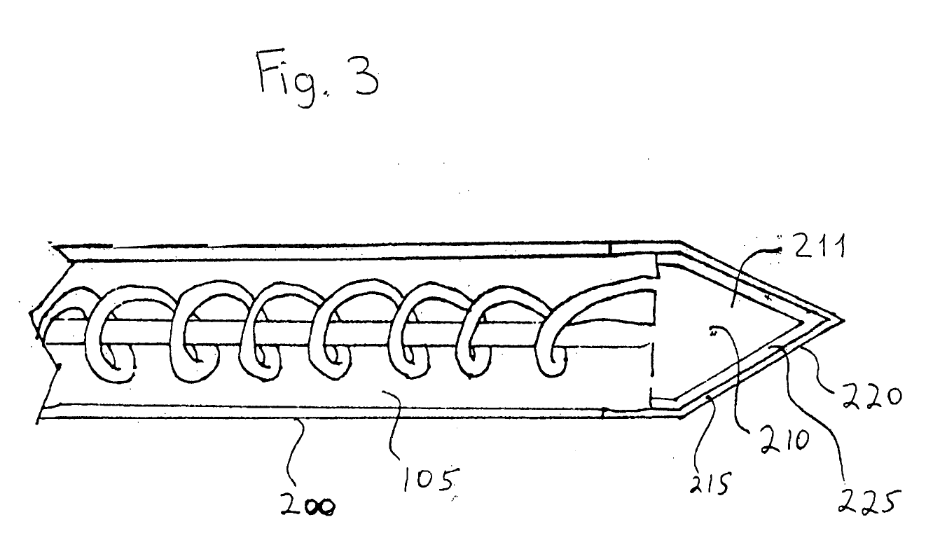

Attention is now drawn to FIG. 3, which presents a simplified schematic of a cryoprobe 200 having a microscopically smooth exterior surface, according to an additional embodiment of the present invention.

FIG. 3 presents a cryoprobe 200 having a cooling surface designed and constructed to minimize adhesion to frozen tissue. Cryoprobe 200 comprises a shaft 105 and a cooling module 210. With respect to its cooling features, cryoprobe 200 may constructed along the lines of cryoprobe 53 presented in FIG. 1, or may be constructed according to any other cryoprobe design producing a cryoprobe operable to cool tissues to cryoablation temperatures. Thus, cooling module 210 may be constructed as described for operating tip 52 of cryoprobe 53, or may be constructed according to alternative methods of cryoprobe construction. In particular, cooling module 210 may be an evaporative cooling module 211, operative to cool cryoprobe 200 by evaporation of a liquefied gas such as liquid nitrogen, liquefied N₂O, liquefied CO₂, or a similar cryogenic fluid.

In a preferred embodiment of the present invention, cryoprobe 200 and cooling module 210 also incorporate features of cryoprobe 100 and cooling module 110 respectively, as those are presented by FIG. 2 and described hereinabove.

Cryoprobe 200 is characterized in that external surface 220 of cooling module 210 is designed and constructed to prevent strong mechanical bonding between ice formed in body tissues and surfaces of cryoprobe 200 by providing an exterior surface 220 characterized by extreme smoothness of construction. Surface 220 is preferably sufficiently smooth to be appearing smooth under magnification to nanometer scale. Surface 220 presents a geometrically simple surface, in those surface 220 does not present ridges, scratches, concavities or other local irregularities. In particular, surface 220 does not provide concavities within which portions ice crystals bonded to frozen portions of tissue might form, and from which concavities such ice crystals would be difficult to dislodge. The structure of surface 220 is to be contrasted to external surfaces of cooling modules of cryoprobes of prior art, which appear smooth to the naked eye yet which, under high magnification, are revealed to contain concavities and various irregular non-smooth forms within which ice crystals may form. Ice crystals forming alongside surface 220 cannot extend into concavities or other irregularities of surface 220 and there form a mechanical bond with surface 220, because surface 220 is smooth and such cavities and irregularities are absent.

Of course, “smoothness” is a relative term, and no real surface is ever absolutely smooth. Hereinafter the term “microscopically smooth” is used to describe the degree of smoothness of surface 220. “Microscopically smooth” refers to smoothness sufficient to substantially prevent mechanical bonding between ice crystals forming next to surface 220 and concavities or other irregularities in surface 220. Preferably, surface 220 is smooth at the atomic or molecular level, or at the nanometer level.

Use of smooth surfaces to reduce adhesion to surfaces has been demonstrated by TOTO Ltd., a Japanese company specializing in plumbing fixtures, and others. TOTO Ltd. sells a toilet having an inner surface of great smoothness, trademarked SanaGloss™, having a markedly reduced tendency to support adhesions as compared to comparable glazed surfaces. Thus, microscopically smooth surfaces have been shown to reduce adhesion of particles to surfaces. The microscopically non-smooth landscape presented by cooling surfaces of conventional cryoprobes present nooks and crannies within which ice crystals form as such probes are operated in cooling. Ice crystals formed within concavities of those non-smooth surfaces bond to ice crystals extending into frozen body tissues, thereby bonding the cryoprobe surface to the freezing tissues. Smoothness of surface 220 of cryoprobe 200 prevents this interaction, consequently probe 200 has a much reduced tendency to mechanically bond to surrounding tissues during freezing of those tissues.

Walls 225 of cooling module 210 are preferably constructed of a strong and highly heat-conducting material such as a metal. Surface 220 may be implemented as a high polished wall 225. However, surface 220 is preferably embodied as a specialized layer 215 applied to the exterior face of wall 225, having a microscopically smooth exterior surface. Layer 215 most preferably comprises non-polar molecules at surface 220, thus combing characteristics of smoothness presented in FIG. 3 with characteristics of non-polar surface presented in FIG. 2.

Optionally, layer 215 may be a ceramic or other material. If a material not highly conductive of heat is used in layer 215, then layer 215 will preferably be thin, as discussed above with respect to layer 115 of cryoprobe 100. Further alternatively, layer 215 may any of a variety of smooth surfaces whose methods of construction are known in the field of nanotechnology.

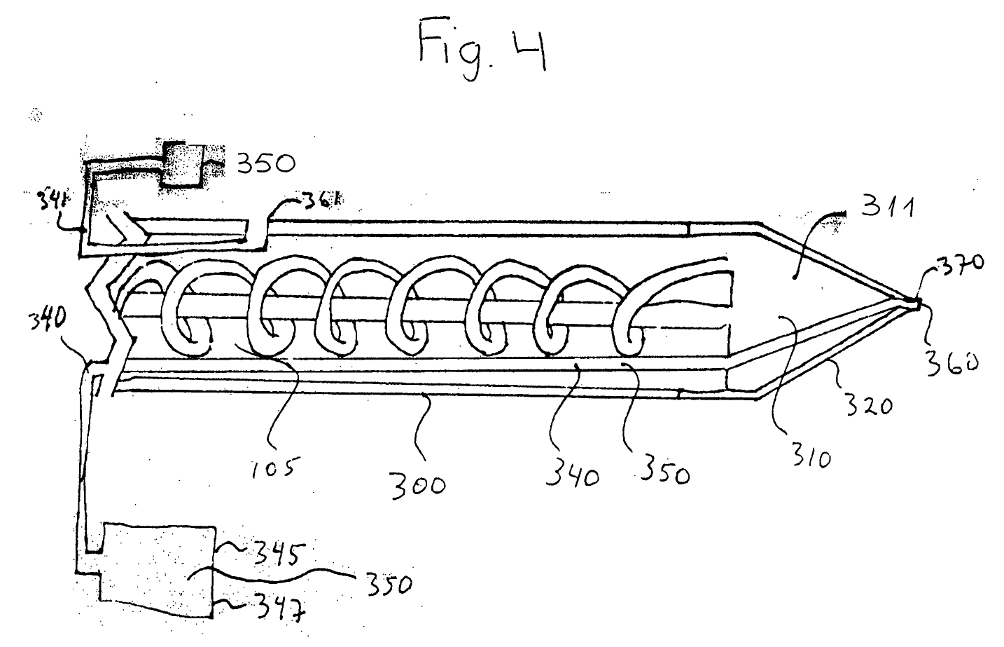

Attention is now drawn to FIG. 4, which presents a simplified schematic of a cryoprobe 300 incorporating a mechanism for delivering a non-polar lubricating substance to an external surface of cryoprobe 300, according to an embodiment of the present invention.

With respect to its cooling features, cryoprobe 300 may constructed along the lines of cryoprobe 53 presented in FIG. 1, or may be constructed according to any other cryoprobe design for making a cryoprobe operable to cool tissues to cryoablation temperatures. Cryoprobe 300 comprises a shaft 105 and a cooling module 310. With respect to its cooling features, cryoprobe 300 may constructed along the lines of cryoprobe 53 presented in FIG. 1, or may be constructed according to any other cryoprobe design producing a cryoprobe operable to cool tissues to cryoablation temperatures. Thus, cooling module 310 may be constructed as described for operating tip 52 of cryoprobe 53, or may be constructed according to alternative methods of cryoprobe construction. In particular, cooling module 310 may be an evaporative cooling module 311, operative to cool cryoprobe 300 by evaporation of a liquefied gas such as liquid nitrogen, liquefied N₂O, liquefied CO₂, or a similar cryogenic fluid.

In a preferred embodiment of the present invention, cryoprobe 300 and cooling module 310 incorporate features of cryoprobe 100 and cooling module 110 respectively, and further incorporate features of cryoprobe 200 and cooling module 210 respectively, as those are presented by FIGS. 2 and 3 and described hereinabove. Thus, in a preferred embodiment of the present invention, cryoprobe 300 comprises an external surface 320 which is preferably non-polar, as described for cryoprobe 100, and preferably microscopically smooth, as described for cryoprobe 200.

In similarity to cryoprobes 100 and 200 presented hereinabove, cryoprobe 300 also has a reduced tendency to form strong bonds with freezing tissues. Cryoprobe 300.comprises a lumen 340 operable to deliver a non-polar lubricating substance 350 to external surface 320 of cooling module 310 of probe 300. Substance 350 may be a biocompatible grease or other biocompatible non-polar substance, and is preferably fluid or semi-fluid both at room temperature and when cooled to low temperatures. Substance 350 preferably has lubricating qualities, appropriate for facilitating movement of cryoprobe 300 through body tissues before, during, and after freezing.

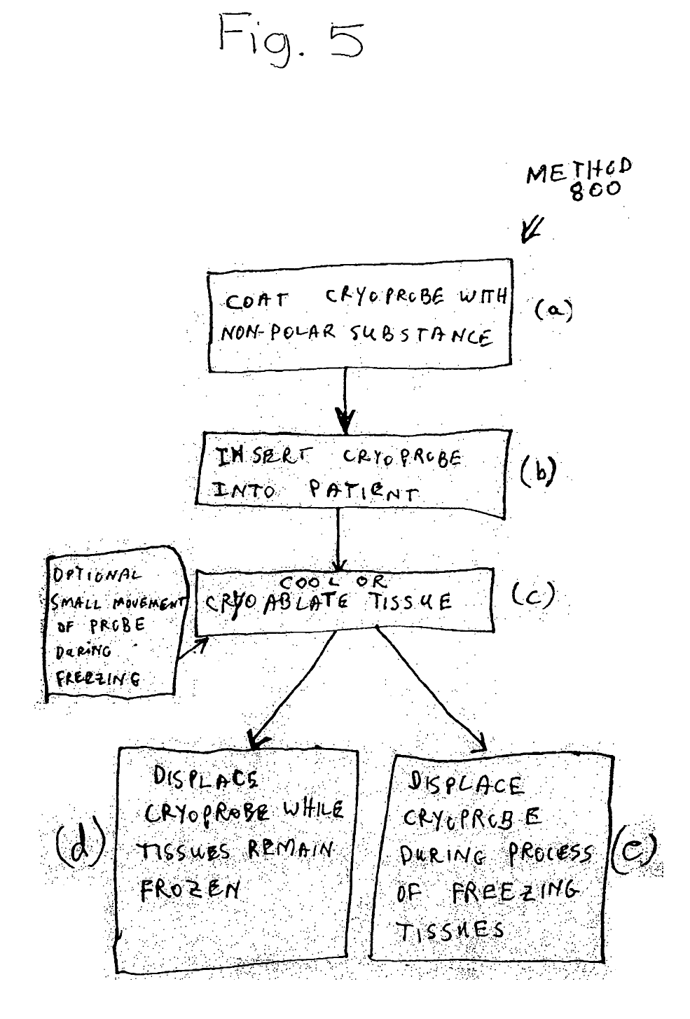

Lumen 340 connects to a lubricating substance source 345 operable to supply substance 350 to lumen 340 under pressure sufficient to enable a desired quantity of substance 350 to be extruded from lumen 340 through an orifice 360 or, in an alternative configuration, through a plurality of orifices 360. In a simple embodiment source 345 is a compressible bag 347 containing substance 350 and connected to lumen 340, such that manual pressure on bag 347 causes substance 350 to move through lumen 340 and out through orifice 360. Orifice 360 is optionally formed as a nozzle 370 shaped to guide substance 350 as it passes through orifice 360 in a manner which facilitates even distribution of substance 350 on surface 320 of probe 300 as probe 300 is inserted into tissues of a patient. Orifice 360 is preferably positioned distally with respect to cooling module 310 of probe 300. In an alternative configuration, one or more additional or alternative orifices 361, also for distributing substance 350 to an external surface of probe 300, may be positioned proximally to cooling module 310 of cryoprobe 300. Orifices 361 may receive substance 350 from lumen 340 or from an independently supplied lumen 341. Uses of orifices 360 and 361 are presented in FIG. 5 and discussed hereinbelow.

Attention is now drawn to FIG. 5, which is a simplified flow chart of a cryotherapy method 800 utilizing a non-polar lubricating substance on a cryoprobe, according to an embodiment of the present invention.

FIG. 5 presents a simple yet effective cryoablation or cryotherapy method, comprising a) coating a cryoprobe (here designated cryoprobe 400, which may be cryoprobe 53, cryoprobe 100, cryoprobe 200, cryoprobe 300, or any other cryoprobe) with a layer of a non-polar lubricating substance such as substance 350 discussed above, b) inserting cryoprobe 400 into a body of a patient, and c) cooling cryoprobe 400 to below freezing temperatures, to treat body tissues. Step (c) may include cooling to cryoablation temperatures, thereby cryoablating body tissues. Step (a) may be practiced before or after step (b), as will be shown hereinbelow.

Additional steps (d) and (e) are optional. These are d) displacing cryoprobe 400 while tissues frozen during step (c) remain frozen (i.e., without first thawing those tissues), and e) navigating (i.e. displacing) cryoprobe 400 while cryoprobe 400 is inserted into tissues of a body of a patient, and while cryoprobe 400 is cooled to cryoablation temperatures.

Step (c) may optionally include imparting small repetitive longitudinal, rotational, or vibratory movements to cryoprobe 400 while ice crystals are being formed adjacent to cryoprobe 400. Formation of strong bonds between such ice crystals and an external surface of cryoprobe 400 will be further discouraged by such movement, which will prevent development of large ice crystal structures bonded to surface features such as concavities of external surfaces of cryoprobe 400.

According to an alternative method, if cryoprobe 400 incorporates features of cryoprobe 100 and/or of cryoprobe 200, then step (a) is optional, and steps (e) and/or (f) may be practiced without step (a), that is, without first coating cryoprobe 400 with an additional layer of non-polar lubricating substance.

Step (a) may be executed by the simple expedient of manually coating cryoprobe 400 with substance 350 prior to insertion of cryoprobe 400 into the body of a patient.

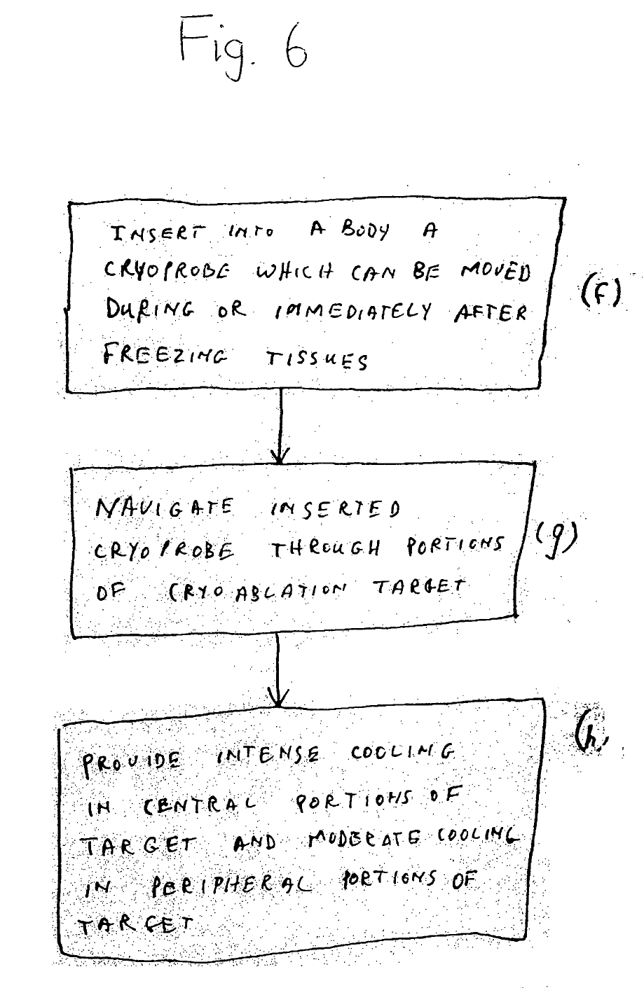

Alternatively (or additionally) step (a) may be executed after step (b), using device and method described hereinabove with respect to FIG. 4. Cryoprobe 300 of FIG. 4 may be used to continuously coat an outer surface of a cryoprobe 300 with a layer of a non-polar lubricating substance during insertion and displacement of cryoprobe within a body of a patient. In a preferred embodiment of method 800, a cryoprobe 300 (preferably also incorporating features of cryoprobes 100 and 200) is inserted into a body of a patient. During insertion of cryoprobe 300, pressure is applied to substance 350 in lumen 340, causing substance 350 to extrude from orifice 360, causing substance 350 to coat external surface 320 and other more proximal surfaces of probe 300 as probe 300 is inserted. Probe 300 is preferably inserted, without cooling, through a selected cryoablation target and up to the most distant point at which cryoablation is desired. Cryoprobe 300 is then cooled to cryocooling or cryoablation temperatures, and, while cooling, is gradually withdrawn through that cryoablation target. In a preferred embodiment presented in detail in FIG. 6 and discussed below, degree of cooling and speed of withdrawal being coordinated so as to produce intense cooling at central portions of the cryoablation target and less intense cooling at peripheral portions of the cryoablation target.

Insertion of cryoprobe 300 creates a channel for movement of cryoprobe 300, which channel is kept open by shaft 105 (not shown) of cryoprobe 300. Cryoprobe 300 is thus enabled to cryoablate while moving through a cryoablation target. Withdrawal of cryoprobe 300 during cooling is preferable to insertion of cryoprobe 300 during cooling since insertion of cryoprobe 300 during cooling might require cryoprobe 300 to penetrate frozen tissue, which would be difficult or impossible. Substance 350 may be supplied through optional orifices 361 of cryoprobe 300 during withdrawal of probe 300, thereby replacing portions of substance 350 that may be wiped away by friction between probe 300 and body tissues as probe 300 is gradually withdrawn.

Step (e) may include optional steps (f) and (g) presented below in discussion of FIG. 6, which steps enable fine control and accurate delimitation of a cryoablation volume.

Attention is now drawn to FIG. 6, which is a simplified flow chart of a procedure for accurately delimiting a cryoablation volume during cryoablation of a cryoablation target in a body of a patient. The steps presented in FIG. 6 may be practiced in the context of methods presented by FIG. 5, or by use of any of the cryoprobes presented herein, or by use of any other cryoprobe 400 operable to be moved within tissues of a body while cooling those tissues to cryoablation temperatures. As mentioned in the background section hereinabove, U.S. patent application Ser. No. 11/066,294 by Zvuloni et al. teaches a method for accurately delimiting a cryoablation volume by using a plurality of cryoprobes to induce intense cooling at central portions of a selected cryoablation target and moderate cooling at peripheral portions of that selected cryoablation target. U.S. patent application Ser. No. 11/066,294 by Zvuloni et al. is incorporated herein by reference. FIG. 6 presents a similar methodology for providing accurate delimitation of a cryoablation target, comprising f) inserting into tissues of a body of a patient a cryoprobe 400 (as defined above) operable to be displaced within that body while being cooled to cryoablation temperatures, g) navigating cryoprobe 400 through portions of a user-specified cryoablation target, and h) utilizing cryoprobe 400 to provide intense cooling when a cooling module of cryoprobe 400 is positioned in central portions of a user-selected cryoablation target, and utilizing cryoprobe 400 to provide moderate cooling when a cooling module of cryoprobe 400 is positioned in peripheral portions of that selected cryoablation target. As noted above, movement of a cooling probe through a cryoablation target is preferably accomplished by inserting that probe to its greatest desired depth within a body, activating the probe in cooling, and gradually withdrawing the probe.

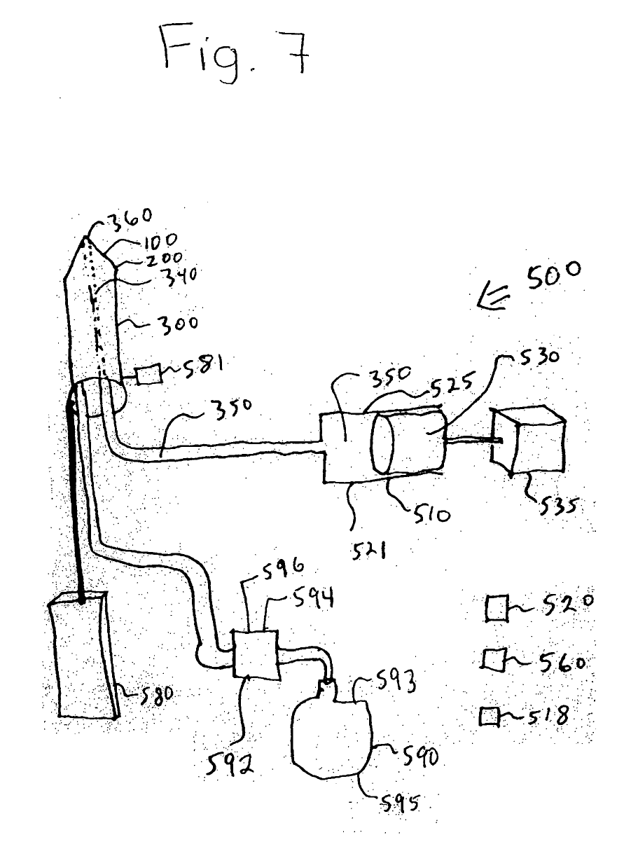

Step (h) may be accomplished by adjusting rate of displacement of cryoprobe 400 through body tissues while cooling, and/or by adjusting rate of cooling of cryoprobe 400 while cryoprobe is continuously or sequentially displaced within a cryoablation target. Selected rates of movement and/or of rates of cooling of cryoprobe 400 are preferably calculated by a control module 560 (presented in FIG. 7) as a function of a detected position of cryoprobe 400 with respect to a user-selected cryoablation target. Thus, a cooling module of a cryoprobe 400 is be cooled to cryoablation temperatures and navigated through a cryoablation target, and may be cooled intensively when positioned within central regions of that target and cooled less intensively when positioned near a border of that target, or may (alternatively or additionally) be displaced more slowly when positioned within central regions of that target and displaced more rapidly (with consequent reduced cooling effect) when positioned near a border of that target. A result of such differential cooling is that interior portions of a cryoablation target are strongly cooled, yet unwanted cooling of healthy tissues exterior to that cryoablation target, yet proximate to it, is reduced.

It is to be noted that methods presented by FIGS. 5 and 6 may be practiced to effect cooling of tissues to temperatures which are below freezing but above cryoablation temperatures, as required by treatment protocols for various clinical conditions.

Attention is now drawn to FIG. 7, which is a simplified schematic of a cryosurgery system designated system 500. System 500 comprises a cryoprobe 300 (preferably incorporating features of cryoprobes 100 and 200 presented above), an optional automatic dispenser 510 of substance 350 for supplying measured amounts of substance 350 to orifice 360 of probe 300, a position sensor 520 operable to detect and characterize movement of probe 300 as probe 300 moves within the body of a patient, a temperature sensor 518 operable to detect and report temperature in or near cryoprobe 300, an optional servomechanism 580 operable to control position and movement of probe 300 within a body of a patient under algorithmic control, a cryogen supply system 590, and a controller 560 for coordinating activities of system 500.

In a preferred embodiment, dispenser 510 is operable to distribute a controlled amount of substance 350, which amount depends on movement of probe 300, the purpose being to provide an appropriate amount of substance 350 to adequately coat probe 300 as probe 300 moves through the body of a patient. In a preferred embodiment dispenser 510 comprises a dispensing module 521 which is an arrangement of a cylinder 525 and piston 530, and a stepper motor 535 causing and controlling movement of piston 530. Controller 560 is operable to receive position and movement information from sensor 520, to calculate desired movements of piston 530 as a function of sensed movement of probe 300, and to command motor 535 to execute those movements, thereby causing an appropriate amount of substance 350 to be extruded onto an external surface of probe 300.

Thus, automatic dispenser 510 is operable to extrude from orifice 360 and/or orifice 361 a quantity of substance 350 selected under algorithmic control and appropriate to provide freedom of movement to probe 300 when probe 300 is operative in cooling and moving in a patient's body. Sensor 520 may also include a detector of degree of force exerted (e.g. by servomechanism 580) to produce a detected degree of movement, that is, sensor 520 may additionally be operable to report resistance to the advancement of probe 300 as probe 300 is displaced within a body.

In a preferred embodiment, system 500 further comprises servomechanism 580. Servomechanism 580 serves to control position and movement of probe 300 within a body of a patient, under command of controller 560, which preferably comprises a user interface for receiving commands of a surgeon. In an embodiment of system 500 incorporating servomechanism 580, information regarding movement of probe 300 within a body of a patient may be gleaned from servomechanism 580 rather than from (or in addition to information from) sensor 520. It is noted that in some contexts servomotor control of movement of probe 300 may be desirable for application of controlled power to movement of probe 300, in that even in absence of adhesion between probe 300 and frozen tissues surrounding probe 300, compression of those tissues against probe 300 is to be expected due to pressure exerted by expansion of ice as tissues freeze.

Servomechanism 580 may be used to impart small repetitive longitudinal, rotational, or vibratory movements to cryoprobe 300 while ice crystals are being formed adjacent to cryoprobe 300, to further reduce bonding between cryoprobe 300 and freezing tissues. In an alternative embodiment, a cryoprobe equipped with an attached small-movement generator 581 such as a manually-controlled motor with appropriate mechanical properties (e.g., an imbalanced flywheel to impart vibratory movement) may be used for this purpose.

In a preferred embodiment, system 500 further comprises cryogen supply system 590 which includes cryogen supply controller 592. For example, if cryoprobe 300 is a cryoprobe cooled by Joule-Thomson cooling, cryogen supply system 590 may be a compressed cooling gas supply 593 operable to supply compressed cooling gas to a Joule-Thomson cooler in cryoprobe 300, and cryogen supply controller 592 may be a remote-control valve 594 responsive to commands from system controller 560 and operable to control flow of cooling gas flowing into or exhausting from cryoprobe 300, thereby controlling cooling of cryoprobe 300 in response to commands from controller 560. Alternatively, cryoprobe 300 may be an evaporative cryoprobe cooled by evaporation of a liquefied gas such as liquid nitrogen, liquefied N₂O, CO₂, or a similar fluid, cryogen supply system 590 may be a liquefied gas supply 595 and cryogen supply controller 592 may be a valve or pump 596 appropriate for controlling delivery of such a cryogen.

Thus, in a preferred embodiment, system 500 comprises cryoprobe 300, which is operable to navigate within tissues of a body while being cooled to below-freezing temperatures, sensor 520 providing real-time data relating to positioning of cryoprobe 300 with respect to a user-selected cryoablation target, sensor 518 providing real-time data relating to temperature of (or near) cryoprobe 300, and controller 560 which is operable to calculate operating parameters for cryoprobe 300 a function of data from sensors 520 and 518, or as a function of position and temperature data from any other sources. System 500 preferably also comprises servomechanism 580, operable to displace cryoprobe 300 within a body of a patient at controlled speed, and cryogen supply 590, operable to control flow of a cooling cryogen (such as compressed gas) to cryoprobe 300 and thus control rate of cooling of cryoprobe 300. Controller 560 is operable to receive temperature data from sensor 518 or another source, to receive data relating to real-time positioning of cryoprobe 300 with respect to a cryoablation target, and to calculate desired operating parameters, such as desired cryoprobe movements and desired cryoprobe temperatures, based on that received data. Controller 560 is further operable to transmit commands to servomechanism 580 to effect those desired cryoprobe movements, and further operable to transmit commands to cryogen supply 590 to effect desired cooling rates and thererby control temperatures in cryoprobe 300. Thus, system 500 is operable to effect a cryoablation operation as described hereinabove with respect to FIGS. 5 and 6, under full or partial algorithmic control.

Thus, if temperature of cryoprobe 300 is held constant, controller 560 can calculate a preferred timed trajectory for cryoprobe 300 within a body as a function of temperature of cryoprobe 300 and as a function of position of cryoprobe 300 in relation to a user-selected cryoablation target. Similarly, if cryoprobe 300 is moved through body tissues at a constant rate, controller 560 can calculate a preferred temperature profile over time for cryoprobe 300, also as a function of positions of cryoprobe 300 with respect to a user-selected cryoablation target. Most preferably, controller 560 will calculate and command both real-time temperature changes and real-time movement changes to achieve optimal accurate ablation of a user-selected cryoablation target.

In an alternative embodiment, system 500 can be implemented using cryoprobe 100 or cryoprobe 200 rather than cryoprobe 300, in which case dispenser 510 would of course be irrelevant.

It is noted that probes 100, 200, 300 and 400, discussed above, may be formed as a cooling expandable balloon catheter similar to that taught by Zvuloni et al. in U.S. Pat. No. 6,875,209, which patent is incorporated herein by reference. Such a balloon catheter may be constructed according to the principles presented herein with respect to cryoprobes 100, 200 and 300 and uses described for cryoprobe 400, thereby providing a cryoplasty balloon catheter operable to move within a blood vessel or other body conduit while cooling body tissues to temperatures below the freezing point of those tissues. To conform to the principles of cryoprobe 200 (a smooth outer surface) such a balloon catheter would presumably be designed and constructed to be inflatable, but not necessarily expandable.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A cryoprobe with reduced tendency to adhere to frozen tissues, comprising one of a group consisting of:
(a) a cooling module having a microscopically smooth external surface;
(b) a cooling module having an external surface comprising non-polar material;
(c) a cooling module coated with a substantially non-polar substance having lubricating qualities at room temperature and when cooled to below-freezing temperatures;
(d) an external orifice through which a biocompatible non-polar substance, delivered to said orifice through communicating with said orifice, may be extruded during movement of said cryoprobe within a body of a patient; and
(e) a mechanical attachment operable to impart small repetitive motions to an inserted cryoprobe while said inserted cryoprobe is cooled to below-freezing temperatures.

2. The cryoprobe of claim 1, having a microscopically smooth external surface comprising non-polar material.

3. The cryoprobe of claim 2, further comprising an external orifice communicating with an internal lumen through which a non-polar substance may be extruded during movement of said cryoprobe within a body of a patient.

4. The cryoprobe of claim 2, further comprising a mechanical attachment operable to impart small repetitive motions to an inserted cryoprobe while said inserted cryoprobe is cooled to below-freezing temperatures.

5. The cryoprobe of claim 1 comprising said mechanical attachment operable to impart small repetitive motions to an inserted cryoprobe while said inserted cryoprobe is cooled to below-freezing temperatures, wherein said repetitive motions are selected from a group consisting of longitudinal movements, rotational movements, and vibratory movements.

6. The cryoprobe of claim 1, comprising a cooling module having an external surface which comprises non-polar material, wherein exposed portions of surface molecules of said external surface are predominantly non-polar.

7. The cryoprobe of claim 1, comprising an external surface which comprises Teflon®.

8. The cryoprobe of claim 1, comprising said orifice, so designed and constructed that said biocompatible non-polar substance extruded through said orifice while said cryoprobe is inserted into a body of a patient at least partially coats an external surface of a cooling module of said cryoprobe.

9. The cryoprobe of claim 1, comprising said orifice, so designed and constructed that said biocompatible non-polar substance extruded through said orifice while said cryoprobe is withdrawn from a body of a patient at least partially coats an external surface of a cooling module of said cryoprobe.

10. The cryoprobe of claim 1, comprising said orifice and wherein said orifice is positioned distally with respect to at least a portion of a cooling module of said cryoprobe.

11. The cryoprobe of claim 1, comprising said orifice wherein said orifice is positioned proximally with respect to at least a portion of a cooling module of said cryoprobe.

12. A cryoprobe with reduced tendency to adhere to frozen tissues, comprising an external surface designed and constructed to form at most weak chemical and mechanical bonding with ice crystals that form in proximity to said cryoprobe when a cooling module of said cryoprobe is cooled to below-freezing temperatures.

13. A system for cryoablation of a user-selected cryoablation target, comprising:
(a) a cryoprobe navigable within tissues of a body while being cooled to below-freezing temperatures;
(b) a first data source providing real-time data relating to positioning of said cryoprobe with respect to said user-selected cryoablation target;
(c) a second data source providing real-time data relating to temperature of said cryoprobe; and
(d) a controller operable to calculate preferred operating parameters for said cryoprobe as a function of data from said first and second data sources.

14. The system of claim 13 further comprising a servomechanism operable to displace said cryoprobe within a body of a patient according to commands issued by said controller.

15. The system of claim 13 further comprising a cryogen supply operable to supply a controlled amount of fluid cryogen to said cryoprobe according to commands issued by said controller.

16. The system of claim 15, wherein said fluid cryogen is a compressed gas and said cryogen supply is operable to supply a controlled flow of said gas to said cryoprobe.

17. The system of claim 15, wherein said cryogen is a liquefied gas and said cryogen supply is operable to control flow of said liquefied gas to said cryoprobe.

18. The system of claim 13, further comprising:
(a) a servomechanism operable to displace said cryoprobe within a body of a patient according to commands issued by said controller; and
(b) a cryogen supply operable to supply controlled quantities of fluid cryogen to said cryoprobe according to commands issued by said controller.

19. The system of claim 18, wherein said controller is operable to calculate and command speed of movement of said cryoprobe within a body of a patient as a function of temperature of said cryoprobe and further as a function of position of said cryoprobe in relation to said user-selected cryoablation target.

20. The system of claim 18, wherein said controller is operable to calculate and command a rate of supply of cryogen to said cryoprobe as a function of position of said cryoprobe with respect to a user-selected cryoablation target, speed of movement of said cryoprobe, and detected temperature of said cryoprobe.

21. A method for cryotreatment of a patient, comprising:
(a) inserting into tissues of a body of said patient a cryoprobe which comprises an external surface designed and constructed to form at most weak bonds with ice crystals that form in proximity to said cryoprobe when a cooling module of said cryoprobe is cooled to below-freezing temperatures; and
(b) displacing said cryoprobe within said tissues while cooling said cryoprobe to below freezing temperatures.

22. The method of claim 21, further comprising using a control module to calculate cooling parameters and movement parameters for said cryoprobe based on a plurality of data streams.

23. The method of claim 22, where said data streams comprise:
(a) data relating to temperature of said cryoprobe; and
(b) data relating to position of said cryoprobe with respect to a user-selected cryoablation target.

24. A balloon catheter sized for insertion into a body conduit and operable to cool a wall of said body conduit, comprising one of a group consisting of:
(a) a cooling module having a microscopically smooth external surface;
(b) a cooling module having an external surface comprising non-polar material;
(c) a cooling module coated with a substantially non-polar substance having lubricating qualities at room temperature and when cooled to below-freezing temperatures;
(d) an external orifice through which a biocompatible non-polar substance, delivered to said orifice through communicating with said orifice, may be extruded during movement of said cryoprobe within a body of a patient; and
(e) a mechanical attachment operable to impart small repetitive motions to an inserted cryoprobe while said inserted cryoprobe is cooled to below-freezing temperatures.
